(19) United States
(12) Patent Application Publication
(10) Pub. No.: US 2011/0087313 A1
(43) Pub. Date: Apr. 14, 2011

(54) Thermal Compression Shirt

(76) Inventors: Brian C. Boatner, Belden, MS (US); Charles L. Boatner, JR., Belden, MS (US)

(21) Appl. No.: 12/632,245

(22) Filed: Dec. 7, 2009

Related U.S. Application Data
(60) Provisional application No. 61/250,907, filed on Oct. 13, 2009.

(51) Int. Cl. A61F 7/00; A41D 1/00

(52) U.S. Cl. 607/112; 607/114; 2/69

(57) ABSTRACT

A thermal garment is tight fitting, often known as a compression shirt, and has a raw or composition rubber sleeve portion, which can be applied to either or both shoulder parts of the garment and/or to either or both elbow parts of the garment, with said rubber portion being covered in fabric and being attached to the garment and replacing that original portion of said garment. The garment with a composition sleeve portion is designed to avoid injury due to extreme playing conditions or improper muscle tension incurred by athletes, by insuring heat and compression to the shoulder joint or to the elbows. The garment also uses heat from the thermal portion of the garment to help in healing the area covered by way of thermotherapy (the use of heat on muscles, joints, tendons, and ligaments which increases blood flow to expedite the healing process).

This application claims the priority of U.S. Provisional Application Ser. No. 61/250,907 entitled “Thermal Compression Shirt” filed on Oct. 13, 2009, the entire contents and substance of which are hereby incorporated in total by reference.

### BACKGROUND OF THE INVENTION

1. Field of the Invention

The invention relates to a compression garment with portions of the garment being used for additional uses other than wear and particularly relates to a compression garment that insures heat and compression to aid in avoiding injuries due to weather or improper muscle tension.

The invention further relates with particularity to a compression garment that emits heat which aids in the healing process of joints by utilizing the added blood flow provided by said form of heat from the garment.

2. Description of Related Art

Sports are enjoyed by many and are an important factor in most individuals in their efforts to maintain a healthy lifestyle. When the term “sports” is mentioned, one most commonly relates the term to activities, where the main outcome of the competitor is to succeed in their physical capabilities and give their best efforts, whether they win or lose. Sports are most often played for fun and just for reason that exercise is a necessity every individual needs to participate in to stay in good physical condition.

One of the main concerns of sports is the risk of injury. Injury can occur in many different ways and on many different levels. Injuries can include anything from, muscle, tendon and ligament tears, strain or physiological stresses, minor pain in parts of the body and even major injuries causing severe pain and discomfort. In addition to the many injuries suffered by athletes today, we can add to the list, bursitis or tendonitis.

Bursitis and tendinitis are both terms used to describe inflammation. Bursitis refers to inflammation of one or more bursae of synovial fluid (thick, stringy fluid found in the cavities of synovial joints) in the body. Bursitis is caused by recurring movement and excessive pressure and most commonly affects the elbows and knees. Bursitis can affect the shoulders as well; shoulder bursitis is more commonly caused by overuse of the shoulder joint and related muscles.

Tendinitis refers to inflammation of the tendon. The inflammation can occur from the overuse of that tendon. The common factors of tendinitis are often related to sports and include excessive forces or loading, poor training techniques, and environmental conditions.

Sports especially those such as baseball, basketball, football, tennis etc. require the constant use of the individuals elbows and shoulders. The constant movement and pressure causes wear and tear on the joints and tendons in athletes' bodies. Another disadvantage some athletes face is weather conditions. Most athletic seasons include times of cold weather conditions, which can cause pain and tenderness in the muscles. Constant use with the addition of cold weather causing pain and tenderness will eventually cause damage to the joints, tendons and muscles. There is a need to contain the shoulders and elbows of the athletes under compression and insure heat to the joints, tendons and muscles. Thus there is a need for a garment that allows the athlete to continue to effectively participate in sports and move about freely without the constraints of big, bulky material and to also insure heat and compression to the athlete's shoulders and elbows during sports related activities.

Injury prevention is important for both professional athletes, beginner athletes trying to make an impression and become professional athletes and even for social athletes whose way of life could be affected by a sports related injury. Thus, the need for products that provide forms of protection to help reduce injuries and in return reduce the disruption injuries cause. In today's society, sports medicine is recognized as a major branch of medicine as sport's injuries are a huge concern and affect more than just the individual.

Injury treatment is often a necessity for athletes, regardless of skill level. There is a need for products that alleviate pain caused by injury that might otherwise force an athlete from withdrawing from competition. The thermal compression garment addresses this dilemma by applying heat to the athlete's affected joint region. Heat has long been used as a method of garnering more blood flow to regions that are covered by the thermal garment. This use of thermotherapy helps in expediting the healing process as well as alleviating pain.

Compression garments are known to be used by athletes to prevent injury. However, these garments do not provide the extra protection of insuring heat to the joints, tendons and muscles.

### SUMMARY OF THE INVENTION

The present invention meets the needs of athletes under cold weather conditions as well as meets the needs of athletes, who in the process of playing sports, utilized wear and tear on the joints in their bodies, especially in the shoulders and elbows of athletes, such as baseball players. The invention is designed to both prevent and alleviate problems related to bursitis or tendonitis, as well as other joint and muscle pain, or such that where inflammation occurs.

One of the proven methods of treating joint pain is the application of heat. Heat needs be utilized while athletes are involved in rigorous athletic activity and not just in the time periods the athletes are sedentary, before or after. With the present invention, the garment allows the athlete to have heat applied, during the activity of the sport, by having the thermal sleeve portion included in the garment; therefore, the athlete is able to comfortably wear the aid in competition.

According to a first broad form of the invention, there is provided improved compression garment, in this case a shirt, for providing compressive forces and heat to one or both of the shoulders and/or elbows on a user comprising:

a thermal material provided heat and increases blood flow to the muscles covered by the material thus relieving pain and tenderness in the muscle. The invention meets the goals set forth by using thermal materials in the shoulder and elbow joint areas which is held together by a less thermal material.

The present invention in its preferred form is a garment utilizing theiuial materials, including but not limited to raw or composition rubber, available in different thicknesses, in the shoulder and/or elbow sleeve portion, which can be applied to either or both shoulder parts of the shirt as well as to the elbows of the shirt with said portion being exposed or covered in fabric. The thermal material is held in place by the material in which the remainder of the shirt is constructed. The materials are joined by various sewing techniques, or by welding.

More importantly, the present invention is designed to avoid injury due to the cold or improper muscle tension incurred by athletes, by insuring heat and compression to the shoulder joint or the elbows. The garment is a tight fitting shirt which custom fits the wearer such that the shirt does not easily shift upon the body of the wearer.

The thermal material provides warmth to the joints while the remaining fabric keeps the thermal material portion secured tightly against the joints, therefore providing both warmth and compression. This in return helps to decrease the injuries incurred by athletes on their shoulder and elbow joints.

The present invention is additionally designed to aid in the healing process of joints by utilizing the added blood flow provided by the heat from the garment. This process is known as thermotherapy which has previously been unavailable to the athlete during the contest. The proposed invention allows the athlete to treat and prevent injury during the activity instead of before or after the activity which left the athlete vulnerable during competition.

In addition to the aforementioned, with respect to the present invention, the thermal compression garment includes the forms of:

The thermal enhancing media is used for heating the joint covered with the media as well as applying pressure and supporting the joint.

The tight fitting material is applied to the rest of the garment which holds the thermal media in place.

The thermal enhancing media is made of one from the group consisting of polyurethane, urethane, PVC, nylon and neoprene.

The compression material being from the group including nylon, polyester, and elastane.

### BRIEF DESCRIPTION OF THE DRAWINGS

The invention will now be described in reference to the following drawings in which:

FIG. 1 shows a perspective view of a garment in the forms of a long-sleeve shirt and short-sleeve shirt in accordance with the subject invention;

FIG. 2 is a front, back and side view of a garment in the form of a long sleeve shirt incorporating a preferred embodiment of the present invention;

FIG. 3 shows a perspective view of the present invention being used in the shoulder sleeves of a short-sleeve garment.

FIG. 4. is a perspective view of the present invention as it is assembled and replaces the sleeve portion of a garment;

### DETAILED DESCRIPTION OF THE INVENTION

During the sequence of this description like numbers will be used to identify like elements according to the different views which illustrate the invention.

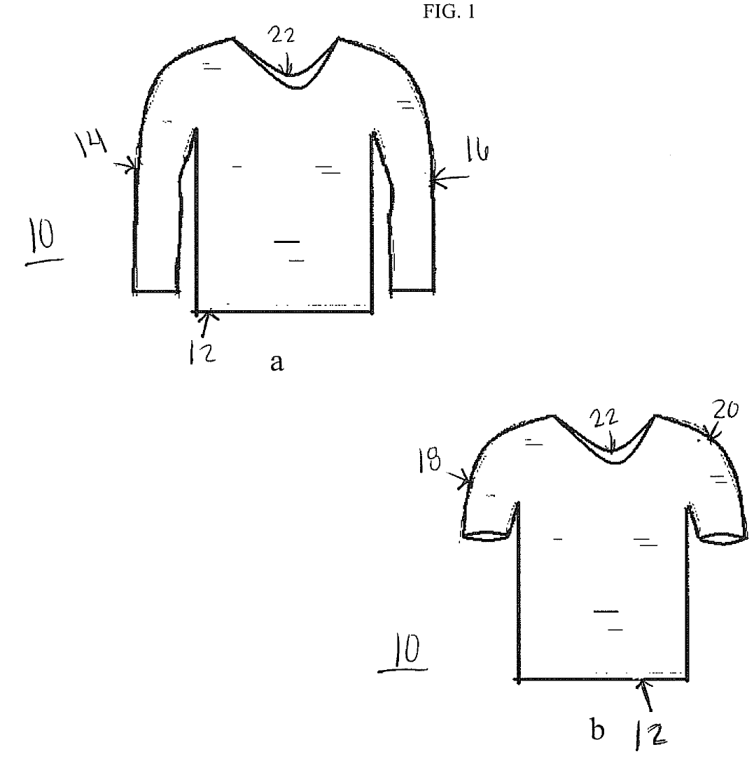

As can be seen in FIG. 1 the invention is a garment 10, and as shown, the garment can be either long-sleeve (a) or short-sleeve (b). The garment has a body portion 12, and can be either long-sleeve 14, 16 or short-sleeve 18, 20 and having a neck opening 22.

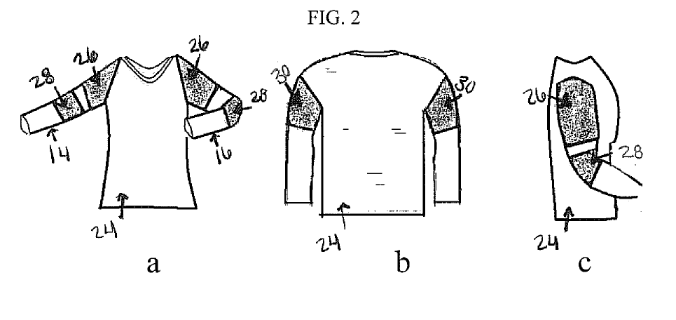

FIG. 2, shows a front view (a) rear view (b) and side view (c) of a long sleeve 14, 16 upper body garment, the garment includes an outer shell 24 made of a compression material which covers the entire garment. In this garment there are four target regions. The front view shows a first black shaded region representing the target region 26 and defining the left and right shoulders with specificity to the humerus (upper arm bone) as well as associated muscles, ligaments and tendons and the biceps brachii. Another black shaded region representing target region 28 on the arms defines the left and right elbow. The rear view shows a black shaded region representing target region 30 on the arms and defines the left and right triceps brachii. The side view is similar to the front view in terms of representation by showing a full side view of the right arm with a black shaded region representing the target region 26 and defining the humerus, as well as associated muscles, ligaments and tendons; the biceps brachii, and a separate black shaded region representing the target region 28 which defines the elbow.

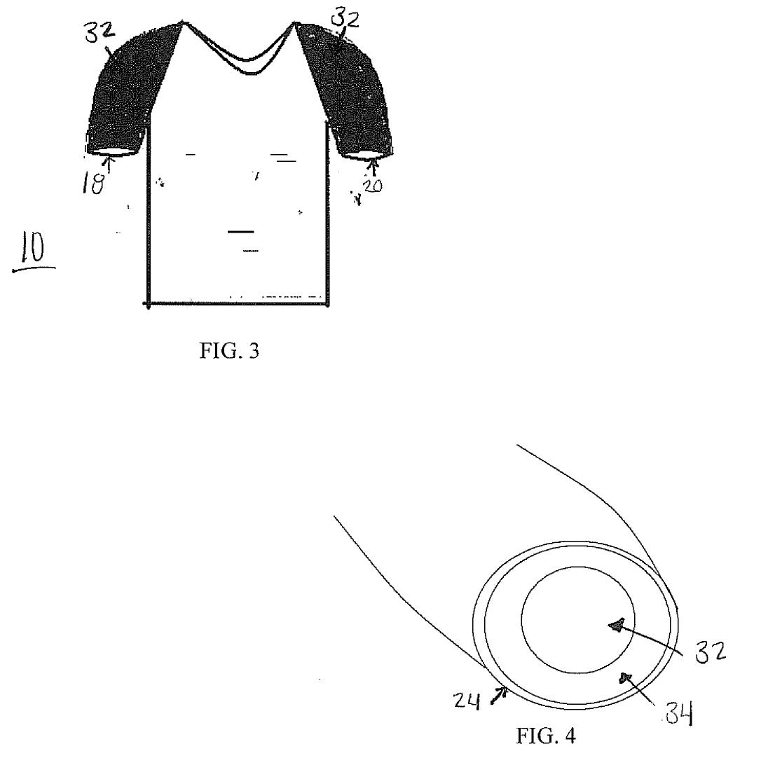

FIG. 3 is one example of how the present invention will be used, as it shows a short-sleeve 18, 20 garment 10 with the shoulder sleeve portion of the garment 10 being replaced with the thermal enhancing media 32 sleeve portion in both the left and right shoulder portions of the shirt. The thermal enhancing media 32 is demonstrated by black being filled into the sleeve portions of the garment 10, which indicates the thermal enhancing media 32 sleeve portion is being used in place of the original sleeve portion of the garment.

FIG. 4 shows a perspective view of a sleeve portion showing the thermal enhancing media 32 being covered in fabric 34 and then being covered in the same compression material as the outer shell 24 of the garment 10. The thermal enhancing media 32 is first bonded or attached by means of sewing or welding to the fabric 34. The thermal enhancing media 32 is available in different thicknesses. The patterns of liner are then stitched into the desired garment shape to fit the individual needs and attached to the garment, which replaces either or both portions of the garments shoulders and/or either or both portions of the garments elbow 28 and looks identical to the conventional outer shell 24 of same garment.

In view of that, a feature and advantage of the present invention is its ability to provide heat to the joints.

Another feature and advantage of the present invention is its ability to permit freedom of movement without sacrifice to the structural integrity of the fabric.

A feature and advantage of the present invention is its use of heat and compression to prevent injury due to extreme playing conditions or joint tension, inflammation and fatigue.

A feature and advantage of the present invention is its use of heat to increase blood flow in regions that are prone to injury.

A feature and advantage of the present invention is its use of heat to reduce or alleviate pain in the tendons or muscles by heating the area covered by the thermal material.

A feature and advantage of the present invention is its tight fitting properties securing the thermal material in the affected region.

A feature and advantage of the present invention is its ability to be worn by athletic participants during sports activities to both prevent and treat injury, where previously this was only done before or after competition, thus leaving the athlete vulnerable to further damage.

A feature and advantage of the present invention is the ability of the athlete to comfortably wear this garment under a uniform and not have constricted movement.

A feature and advantage of the present invention is the thermal portion is attached directly to the garment, replacing the original specific portion of that garment.

A feature and advantage of the present invention is the option of customization of the thermal material depending on the individual needs of the athlete.

A feature and advantage of the present invention is its durability.

A feature and advantage of the present invention is its light weight.

While the invention has been described with reference to the preferred embodiment thereof, it will be appreciated by those of ordinary skill in the art that modifications can be made to the structure and elements of the invention without departing from the spirit and scope of the invention as a whole.

The invention claimed is:

1. A thermal compression garment comprising a piece of clothing having at least one region of thermal enhancing media, wherein the region is at a specific location and attached to the clothing;
wherein the region is comprised of thermal enhancing media enclosed in a fabric, and wherein the entirety of the enclosure is attached directly to the clothing.

2. The garment according to claim 1, wherein the thermal enhancing media is made up of one from the group consisting of polyurethane, urethane, PVC, nylon and neoprene.

3. The garment according to claim 1, wherein the material for enclosure is of spandex fabric and the clothing material is compression being from the group including nylon, polyester, and elastane.

4. The garment according to claim 1, wherein the attachment of the thermal enhancing media to the fabric for enclosure is done using various sewing techniques.

5. The garment according to claim 1, wherein the attachment of the thermal enhancing media enclosed in fabric is attached to a region in the clothing, replacing same said region of the original clothing.

6. The garment according to claim 1, wherein the thickness of the thermal enhancing media varies and is based upon individual need.

7. The garment according to claim 1, wherein the garment is a shirt.

8. The garment according to claim 7, wherein a thermal enhancing media enclosed in fabric is attached to the garment and being the left shoulder region of the shirt, replacing the original left shoulder region in the sleeve of the garment.

9. The garment according to claim 7, wherein a thermal enhancing media enclosed in fabric is attached to the garment and being the right shoulder region of the shirt, replacing the original right shoulder region in the sleeve of the garment.

10. The garment according to claim 7, wherein a thermal enhancing media enclosed in fabric is attached to the garment and being the left elbow region of the shirt, replacing the original left elbow region in the sleeve of the garment.

11. The garment according to claim 7, wherein a thermal enhancing media enclosed in fabric is attached to the garment and being the right elbow region of the shirt, replacing the original right elbow region in the sleeve of the garment.
